(19) United States
(12) Patent Application Publication
(10) Pub. No.: US 2021/0174897 A1
(43) Pub. Date: Jun. 10, 2021

(54) METHOD FOR GENERATING FREQUENCY DISTRIBUTION OF BACKGROUND ALLELE IN SEQUENCING DATA OBTAINED FROM ACELLULAR NUCLEIC ACID, AND METHOD FOR DETECTING MUTATION FROM ACELLULAR NUCLEIC ACID USING SAME

(71) Applicant: GENINUS INC., Seoul (KR)

(72) Inventors: Woong Yang PARK, Seocho-gu, Seoul (KR); Dong Hyun PARK, Gangnam-gu, Seoul (KR); Dae Soon SON, Gwanak-gu, Seoul (KR)

(21) Appl. No.: 16/616,773

(22) PCT Filed: Apr. 17, 2018
(86) PCT No.: PCT/KR2018/004405

§ 371 Date: Nov. 25, 2019

(30) Foreign Application Priority Data
May 24, 2017 (KR) 10-2017-0064387

(51) Int. Cl. G16B 25/10; G16B 40/00; G16B 20/20

(52) U.S. Cl. CPC G16B 25/10; G16B 20/20; G16B 40/00

(57) ABSTRACT

Provided are a method for generating a distribution of background allele frequency in sequencing data obtained from a cell-free nucleic acid, a frequency distribution matrix of background alleles obtained by the method, and a method of detecting a variation in the cell-free nucleic acid using the matrix. According to the method, to remove germline variations, sequencing data of a nucleic acid isolated from a cell of a test subject itself may be used to generate a distribution of background allele frequency in the sequencing data obtained from the cell-free nucleic acid, and thus there are advantages in terms of reducing costs and time.

### TECHNICAL FIELD

The preset disclosure relates to a method of and a device for generating a distribution of background allele frequency in sequencing data obtained from a cell-free nucleic acid, a frequency distribution matrix of background alleles obtained by the method, and a method of and a device for detecting a variation in the cell-free nucleic acid using the matrix.

### BACKGROUND ART

A genome refers to all the genetic information possessed by an organism. For sequencing or sequence analysis of the genome of an individual, various techniques such as DNA chips, next-generation sequencing (NGS), next next-generation sequencing (NNGS), etc. have been developed. NGS is widely used for research and diagnostic purposes. NGS varies depending on the type of equipment, but it may be largely divided into three stages: sample collection; library preparation; and nucleic acid sequencing. After nucleic acid sequencing, genetic variations are detected based on the produced sequencing data.

Sequencing error rates of current NGS reach 0.1% to 1% due to errors caused by polymerase during polymerase chain reaction (PCR), errors caused by fluorescence detection during nucleic acid sequencing, etc. These errors have a problem of inhibiting the detection of rare variations that occur at frequencies below the sequencing error rate. To overcome this problem, it is necessary to increase the number of samples that need variation analysis during sequencing, or to perform sequencing several times. However, this method requires very high sequencing costs and a large amount of samples.

Meanwhile, in a method of preparing a library, a method of detecting rare variations by remarkably increasing the number of reads by improving an adapter sequence and/or a barcode sequence is known (Korean Patent Publication No. 10-2016-0141680A). However, little is known about methods capable of reducing errors that may occur in stages other than library preparation and sequencing stages.

Accordingly, there is a demand for a method capable of accurately detecting rare variations while minimizing expenditure.

### DESCRIPTION OF EMBODIMENTS

### Technical Problem

An aspect provides a method of generating a distribution of background allele frequency in sequencing data obtained from a cell-free nucleic acid.

Another aspect provides a device for generating a distribution of background allele frequency in sequencing data obtained from a cell-free nucleic acid.

Still another aspect provides a frequency distribution matrix of background alleles in sequencing data obtained from a cell-free nucleic acid.

Still another aspect provides a method of detecting variations in a cell-free nucleic acid.

Still another aspect provides a device for detecting variations in a cell-free nucleic acid.

### Solution to Problem

An aspect provides a method of generating a distribution of background allele frequency in sequencing data obtained from a cell-free nucleic acid.

The method comprises: obtaining first sequencing data of one or more positions on a chromosome from the cell-free nucleic acid; obtaining second sequencing data of one or more positions on the chromosome from a nucleic acid isolated from a cell; generating a distribution of background allele frequency at one or more positions on the chromosome, based on the second sequencing data; and estimating the distribution of background allele frequency in the first sequencing data using the distribution of background allele frequency.

The method may include obtaining first sequencing data of one or more positions on the chromosome from a cell-free nucleic acid; and obtaining second sequencing data of one or more positions on the chromosome from a nucleic acid isolated from a cell. The method may include obtaining sequencing data of one or more positions on the chromosomes from a nucleic acid isolated from a cell and a cell-free nucleic acid. The obtaining the first sequencing data and the obtaining the second sequencing data may be performed simultaneously or sequentially.

The “sequencing or sequence analysis” may be next generation sequencing (NGS). The NGS may be used interchangeably with massive parallel sequencing or second-generation sequencing. The NGS is a technique for multiple simultaneous sequencing of a large amount of nucleic acid fragments, in which the full-length genome is fragmented into chip-based and polymerase chain reaction (PCR)-based paired ends, and the fragments may be subjected to ultra-high-speed sequencing, based on hybridization. The NGS may include NGS-based targeted sequencing, targeted deep sequencing, or panel sequencing. The NGS may be performed by, for example, 454 platform (Roche), GS FLX titanium, Illumina MiSeq, Illumina HiSeq, Illumina HiSeq 2500, Illumina Genome Analyzer, Solexa platform, SOLiD System (Applied Biosystems), Ion Proton (Life Technologies), Complete Genomics, Helicos Biosciences Heliscope, Pacific Biosciences' Single-molecule real-time sequencing (SMRT™) technology, or combination thereof.

The sequencing data refers to data obtained by the sequencing or sequence analysis, and may include alleles and frequencies thereof at one or more positions or all positions on the chromosome to be sequenced. The first sequencing data refers to sequencing data obtained from one or more positions on the chromosome from a cell-free nucleic acid, and the second sequencing data refers to sequencing data obtained from one or more positions on the chromosome from a nucleic acid isolated from a cell. The sequencing data may be obtained from, for example, data of binary version of SAM (BAM) format and/or Sequence Alignment/Map (SAM) format. The BAM format and/or the SAM format may be commonly those used as a format for describing data of short reads. The data of the BAM format and/or the SAM format may include text data of FLAG or compact idiosyncratic gapped alignment report (CIGAR) string representing start points of reads, direction of reads, mapping quality, and order of alignment. Various supporting reads may be obtained by generating various alignment pairs.

The nucleic acid may be genome or a fragment thereof. The term “genome” refers to chromosome, chromatin, or the entirety of genes. The nucleic acid may be deoxyribonucleic acid (DNA), ribonucleic acid (RNA), or a combination thereof.

The nucleic acid isolated from a cell may be a nucleic acid isolated from a cell or a cell line. The nucleic acid isolated from a cell may be isolated from a cell present in blood, serum, urine, saliva, mucous secretions, sputum, feces, tears, or a combination thereof. The nucleic acid isolated from a cell may be isolated from a blood cell, an oral epithelial cell, a hair follicle cell, a skin fibroblast, or a combination thereof. The blood cell may be, for example, leukocyte, specifically, peripheral blood leukocyte (PBL), and more specifically, a peripheral blood mononudear cell (PBMC) and/or a polymorphonuclear leukocyte (PML), including a peripheral blood monocyte and/or a peripheral blood lymphocyte. The cell-free nucleic acid (cf nucleic acid) may be a nucleic acid released by a cell. The cell-free nucleic acid may be present in blood, plasma, serum, urine, saliva, mucous secretions, sputum, feces, tears, or a combination thereof. The cell-free nucleic acid may be a circulating tumor nucleic acid (ct nucleic acid). The cell-free nucleic acid may be, for example, cell-free DNA (cfDNA). A method of extracting or isolating the nucleic acid may be performed by a method known to those skilled in the art.

The one or more positions on the chromosome refer to positions on the chromosome which is examined to detect whether genetic variations exist or not. The position on the chromosome may be, for example, a position at which a variation is predicted to exist and which may be a target region for targeted sequencing. The allele at each position on the chromosome, allele frequency, and allele frequency distribution may be obtained from the sequencing data of one or more positions on the chromosome. The position on the chromosome may be expressed by the chromosome numbering, for example, chr8:19, 939,070-19,967,258, or 17p 13.1.

The method may include generating a distribution of background allele frequency at one or more positions on the chromosome, based on the second sequencing data. The method may include generating a distribution of background allele frequency at one or more positions on the chromosome, based on the sequencing data of a nucleic acid isolated from a cell.

The background allele may be (1) a non-reference allele, (2) not an allele due to germline variation, and/or (3) not a genotype of a subject itself. The background allele may be used interchangeably with a background allele error. The background allele may be a base misinterpreted by technical errors, for example, a base misinterpreted by errors that occur in the overall process of performing sequencing.

The background allele frequency means background allele detection frequency, background allele generation frequency, a background allele error rate, or a background allele error occurrence rate. The distribution of background allele frequency means a range including the minimum and maximum of the background allele detection frequency. The background allele frequency may be calculated by counting the number of each allele.

The data of reference genome may be obtained from a database already known in the art, such as National Center for Biotechnology Information (NCBI), Gene Expression Omnibus (GEO), Food and Drug Administration (FDA), My Cancer Genome, The Cancer Genome Atlas (TCGA), etc., or obtained from a biological sample of a control group, i.e., a normal person. The normal person may be a healthy person in which a specific disease, for example, a tumor, is not found. The reference genome may be a human reference genome, and may be hg18 or hg19.

The method may include estimating the distribution of background allele frequency in the first sequencing data using the distribution of background allele frequency. The above step may include applying the distribution of background allele frequency generated from the nucleic acid isolated from the cell to the distribution of background allele frequency in the sequencing data obtained from the cell-free nucleic acid. FIG. 4 is a flow chart showing a method including removing germline variations using sequencing data obtained from peripheral blood leukocytes of a patient who is a test subject, generating a frequency distribution matrix of background allele errors, and detecting variations in cell-free nucleic acid in the plasma. Generally, when a variation is detected in a cell-free nucleic acid derived from a test subject, a distribution of background allele frequency at one or more positions on the chromosome is generated, based on sequencing data obtained from a nucleic acid of a control healthy person, and any allele frequency in the sequencing data of the cell-free nucleic acid derived from the test subject is compared with the distribution of background allele frequency generated from the nucleic acid of the healthy person. If greater, it is determined that the allele is a significant variant, otherwise, it is determined that the allele is not a significant variant. In this case, to detect whether or not the test subject has variations, the sequencing data obtained from the nucleic acid of the control normal person are required, and thus additional time and cost are consumed. However, to remove a germline variation, processes of obtaining the sequencing data of the nucleic acid isolated from the test subject-derived cell and detecting variations are required. According to the above method, the distribution of background allele frequency in the sequencing data of the cell-free nucleic acid may be generated, based on the sequencing data of the nucleic acid isolated from the cell of the test subject itself, and thus there are advantages in terms of reducing costs and time.

The method may include performing fragmentation of the nucleic acid isolated from the cell, prior to obtaining the second sequencing data. The fragmentation may be physical, chemical, thermal, optical, ultrasonic, or enzymatic cleavage of the genome. For example, the chemical cleavage may be cleavage by reacting with restriction enzymes. The ultrasonic cleavage may be applying ultrasonic wave. The ultrasonic cleavage may be applying ultrasonic waves of about 50 W to about 160 W, about 60 W to about 160 W, about 70 W to about 160 W, about 80 W to about 160 W, about 90 W to about 160 W, or about 100 W to about 150 W. The ultrasonic cleavage may be applying ultrasonic waves for about 10 seconds to about 300 seconds, about 20 seconds to about 250 seconds, about 20 seconds to about 200 seconds, about 30 seconds to about 150 seconds, about 40 seconds to about 100 seconds, or about 45 seconds to about 90 seconds.

The fragmentation may be to perform cleavage while reducing physical, chemical, thermal, optical, ultrasonic, or enzymatic energy which is applied to the genome. When the energy is above a predetermined threshold, nucleic acid fragments form base pairs, in which a purine base forms a base pair with another purine base, or a pyrimidine base forms a base pair with another pyrimidine base. For example, when the energy applied to the fragmentation is excessive, oxidative damage occurs in guanine (G), which is then converted to thymine (T), and the converted thymine (T) may form a base pair with adenosine (A). To prevent the formation of such erroneous base pairs, oxidative damage may be reduced by reducing the energy applied during fragmentation. When fragmentation is performed while reducing the physical, chemical, thermal, optical, ultrasonic, or enzymatic energy so that the sizes of the nucleic acid fragments become larger than 200 bp, oxidative damage may be reduced to prevent the formation of erroneous base pairs. As a result, since the distribution of background allele frequency in the sequencing data obtained from the cell-free nucleic acid and the distribution of background allele frequency in the sequencing data obtained from the nucleic acid isolated from the cell may exhibit a similar pattern, the distribution of background allele frequency in the sequencing data obtained from the nucleic acid isolated from the cell may be estimated and applied to the distribution of background allele frequency in the sequencing data obtained from the cell-free nucleic acid.

The method may further include size-sorting the nucleic acid fragments. The sizes of the nucleic acid fragments may be 200 bp or more. The sizes of the nucleic acid fragments may be 200 bp or more, 250 bp or more, 300 bp or more, 310 bp or more, 320 bp or more, 330 bp or more, 340 bp or more, 350 bp or more, 360 bp or more, 370 bp or more, 380 bp or more, 390 bp or more, 400 bp or more, 410 bp or more, 420 bp or more, 430 bp or more, 440 bp or more, 450 bp or more, 460 bp or more, 470 bp or more, 480 bp or more, 490 bp or more, or 500 bp or more. The size of the cell-free nucleic acid is generally 150 bp to 200 bp, and the sizes of the fragments of the nucleic acid isolated from the cell are 200 bp or more, for example, larger than the size of the cell-free nucleic acid.

The nucleic acid isolated from the cell and the cell-free nucleic acid may be derived from the same subject or different subjects. As described above, the distribution of background allele frequency in the sequencing data obtained from the cell-free nucleic acid may be generated, based on the sequencing data obtained from a nucleic acid of the test subject itself or a different subject belonging to the same species. The subject may be a subject having a disease, a subject having a tumor, a normal person, or a combination thereof. The subject may be a mammal including a human, a cow, a horse, a pig, a sheep, a goat, a dog, a cat, and a rodent.

Another aspect provides a frequency distribution matrix of background allele in the sequencing data obtained from the cell-free nucleic acid according to the above method. The frequency distribution matrix of the background allele may be an integrated representation of alleles at one or more positions or all positions on the chromosome to be sequenced, allele frequency, and allele frequency distribution.

Still another aspect provides a method of detecting variations in the cell-free nucleic acid.

The method includes obtaining first sequencing data of one or more positions on the chromosome from the cell-free nucleic acid; obtaining second sequencing data of one or more positions on the chromosome from a nucleic acid isolated from a cell; generating a distribution of background allele frequency at one or more positions on the chromosome, based on the second sequencing data; and detecting variations by comparing any allele frequency at one or more positions on the chromosome in the first sequencing data with the distribution of background allele frequency at positions corresponding thereto.

The obtaining the first sequencing data of one or more positions on the chromosome from the cell-free nucleic acid; the obtaining the second sequencing data of one or more positions on the chromosome from the nucleic acid isolated from the cell; and the generating the distribution of background allele frequency at one or more positions on the chromosome, based on the second sequencing data, are the same as described above.

The variation means a genetic variation as a structural variation of the chromosome, and may include a common variation (a common and/or polygenic variant), a rare variation (a rare variant), or a combination thereof. The genetic variation may be an indicator or a marker explaining the risk of a disease or the incidence of a disease. The rare variation may mean a variation having variant allele frequency of 5% or less, 4.5% or less, 4% or less, 3.5% or less, 3% or less, 2.5% or less, 2% or less, 1.5% or less, 1% or less, 0.9% or less, 0.8% or less, 0.7% or less, 0.6% or less, 0.5% or less, 0.4% or less, 0.2% or less, 0.1% or less, 0.09% or less, 0.08% or less, 0.07% or less, 0.06% or less, 0.05% or less, 0.04% or less, 0.03% or less, 0.02% or less, or 0.01% or less.

The variation may include alteration of a base, a nucleotide, a polynucleotide, or a nucleic acid, and may include substitution, insertion, duplication, deletion, or insertion and deletion (‘InDel’) of a base, a nucleotide, a polynucleotide, or a nucleic acid, etc. The variation may be a single nucleotide variant (SNV), a single nucleotide polymorphism (SNP), or a combination thereof.

The method may include detecting variations by comparing any allele frequency at one or more positions on the chromosome in the first sequencing data with the distribution of background allele frequency at positions corresponding thereto.

The method may include determining that the allele is a significant variant when any allele frequency at one or more positions on the chromosome in the first sequencing data is larger than the distribution of background allele frequency at the positions corresponding thereto, and determining that the allele is not a significant variant when any allele frequency at one or more positions on the chromosome in the first sequencing data is smaller than or equal to the distribution of background allele frequency at the positions corresponding thereto.

In other words, the method may include determining that the allele is a significant variant when any allele frequency at one or more positions on the chromosome in the sequencing data obtained from the cell-free nucleic acid is larger than the distribution of background allele frequency at the positions corresponding thereto in the sequencing data obtained from the nucleic acid isolated from the cell, and otherwise, determining that the allele is not a significant variant. According to the method, it is possible to accurately discriminate whether any allele frequency at one or more positions on the chromosome in the sequencing data obtained from the cell-free nucleic acid is a significant variant or an error.

The method of generating the distribution of background allele frequency in the sequencing data obtained from the cell-free nucleic acid according to an aspect and the method of detecting variations in the cell-free nucleic acid may be applied to a personalized diagnostic or therapeutic method or a precise treatment method. Specifically, the present disclosure provides a personalized diagnostic or therapeutic method or a precise treatment method, the method further including performing personalized diagnosis or treatment (e.g., precise treatment) depending on the kind of the detected variation, after generating the distribution of background allele frequency or detecting nucleic acid variations by the above method.

Still another aspect provides a device for generating the distribution of background allele frequency in the sequencing data obtained from the cell-free nucleic acid.

The device may include a memory; and a processor.

The memory includes memory chips such as random access memory (RAM), read only memory (ROM), etc., or storages such as hard disk drive (HDD), solid state drive (SSD), etc. as hardware for storing data to be processed and processed results in a computing device. In other words, the memory may store the first sequencing data, the second sequencing data, and the distribution data of background allele frequency which are obtained by the processor.

The processor may include a first acquiring unit that is configured to acquire the first sequencing data of one or more positions on the chromosome from the cell-free nucleic acid; a second acquiring unit that is configured to acquire the second sequencing data of one or more positions on the chromosome from the nucleic acid isolated from the cell; a generating unit that is configured to generate the distribution of background allele frequency at one or more positions on the chromosome, based on the second sequencing data; and an estimating unit that is configured to estimate the distribution of background allele frequency in the first sequencing data using the distribution of background allele frequency.

The acquiring unit of the processor may be to acquire from a sequencing or sequence analysis device.

Further, the processor is performed by the method as mentioned above.

The processor is a module implemented with one or more processing units, and the processor may be implemented in a combination of a microprocessor having an array of multiple logic gates and a memory module storing a program that may be executed on the microprocessor. The processor may be implemented in the form of a module of an application program.

Still another aspect provides a device for detecting a variation in the cell-free nucleic acid.

The device includes a memory; and a processor.

The memory is the same as described above.

The processor may include a first acquiring unit that is configured to acquire the first sequencing data of one or more positions on the chromosome from the cell-free nucleic acid; a second acquiring unit that is configured to acquire the second sequencing data of one or more positions on the chromosome from the nucleic acid isolated from the cell; a generating unit that is configured to generate the distribution of background allele frequency at one or more positions on the chromosome, based on the second sequencing data; and a detecting unit that is configured to detect variations by comparing any allele frequency at one or more positions on the chromosome in the first sequencing data with the distribution of background allele frequency at the positions corresponding thereto.

### Advantageous Effects of Disclosure

According to a method of and a device for generating a distribution of background allele frequency in sequencing data obtained from a cell-free nucleic acid, a frequency distribution matrix of background alleles obtained by the method, and a method of and a device for detecting a variation in the cell-free nucleic acid using the matrix, a process of obtaining sequencing data from blood, a cell, or a cell-free nucleic acid of a normal person may be omitted, and therefore, there are advantages in terms of reducing costs and time. Further, when variations are detected in the cell-free nucleic acid using the distribution of background allele frequency, reliability and accuracy of the detection result may be improved in detecting a very small amount of variation.

### BRIEF DESCRIPTION OF DRAWINGS

FIG. 1A shows Phred base quality score distribution of each of background allele bases and total allele bases in PBL DNA samples and plasma DNA samples, respectively; FIG. 1B shows base quality score distribution of each of reference allele bases and background allele bases in PBL DNA samples, after removal of bases with a quality score <30; FIG. 1C shows base quality score distribution of each of reference allele bases and background allele bases in plasma DNA samples, after removal of bases with a quality score <30;

FIG. 2A shows background allele frequencies from 19 plasma DNA samples and 19 PBL DNA samples, i.e., mean error rates of background alleles in each sample; FIG. 2B shows frequency of background allele error-free positions in plasma DNA samples and PBL DNA samples; FIG. 2C shows a distribution of background allele frequencies across 12 base substitution classes in plasma DNA samples and PBL DNA samples, wherein the y-axis represents the background allele frequency of each substitution class in the pre-treatment PBL DNA samples and plasma DNA samples; FIGS. 2D and 2E show background allele error rates for 12 base substitution classes in plasma DNA samples and PBL DNA samples, wherein error bars indicate standard deviation;

FIG. 3A shows background allele error rates from sequencing data generated using genomic DNA fragments as input DNA, wherein the genomic DNA fragments were obtained by fragmentation under various fragmentation conditions; FIG. 3B shows detailed conditions of the fragmentation conditions used in FIG. 3A and the sizes of the resulting fragments; and

FIG. 4 shows a flow chart showing a method including removing germline variations using sequencing data obtained from peripheral blood leukocytes of a patient who is a test subject, generating a frequency distribution matrix of background allele errors, and detecting variations in cell-free nucleic acid in the plasma.

### MODE OF DISCLOSURE

Hereinafter, the present disclosure will be described in more detail with reference to embodiments. However, these embodiments are for illustrating the present disclosure, and the scope of the present disclosure is not limited to these embodiments.

### Example 1. Generation of Distribution of Background Allele Frequency in Sequencing Data Obtained from Cell-Free Nucleic Acid

1. Generation and comparison of distributions of background allele frequencies in sequencing data obtained from nucleic acid isolated from cell and cell-free nucleic acid

(1) Plasma and Peripheral Blood Lymphocyte (PBL) Collection and DNA Extraction

Blood was collected from two healthy normal persons and 17 patients with pancreatic cancer. The blood samples were collected in cell-free DNAM BCT tubes (Streck Inc., Omaha, Nebr., U.S.A.). The collected blood samples were processed within 6 hours of collection via three graded centrifugation steps of at 840 g for 10 minutes, at 1040 g for 10 minutes, and 5000 g for 10 minutes at 25° C. Peripheral blood lymphocytes (PBLs) were drawn from the first step of centrifugation. Plasma was transferred to new tubes at each step of centrifugation. Plasma samples and PBL samples were stored at −80° C. until cell-free DNA (cfDNA) extraction.

Germline DNAs were isolated from peripheral blood mononuclear cells (PBMCs) using a QIAamp DNA mini prep kit (Qiagen, Santa Clarita, Calif., U.S.A.). Circulating DNAs were extracted from 1 mL to 5 mL of plasma using a QIAamp circulating nucleic acid kit (Qiagen). DNA concentration and purity were assessed by a PicoGreen fluorescence assay using a Qubit 2.0 fluorometer (Life Technologies, Grand Island, N.Y., U.S.A.) with a Qubit dsDNA HS assay kit and a BR assay kit (Thermo Fisher Scientific, Waltham, Mass., U.S.A.). DNA concentration and purity were quantified using a Nanodrop 8000 UV-Vis spectrometer (Thermo Fisher Scientific) and a Picogreen fluorescence assay. The fragment size distribution was measured using a 2200 TapeStation Instrument (Agilent Technologies, Santa Clara, Calif., U.S.A.) and real-time PCR Mx3005p (Agilent Technologies) according to the manufacturer's instructions.

(2) Library Preparation

Genomic DNAs from PBL samples were sonicated using a Covaris S220 (Covaris Inc., Woburn, Mass., U.S.A.) under conditions of a duty factor of 10%, a peak incident power of 175 W, and 200 cycles/burst for 6 minutes according to the manufacturer's instructions. DNAs from plasma samples were prepared without fragmentation.

To construct sequencing libraries, 200 ng of PBL DNA sample and 37.3 ng of plasma DNA sample were used. The libraries for PBL and plasma DNA samples were constructed using a KAPA Hyper Prep Kit (Kapa Biosystems, Woburn, Mass., U.S.A.). End repair, adenosine tailing (A tailing), and adapter ligation were performed for each DNA according to the manufacturer's instructions. Polymerase chain reactions were performed for amplification. At this time, a purification step was carried out using AMPure beads (Beckman Coulter, Ind., U.S.A.) after each procedure. Adaptor ligation was performed using a pre-indexed PentAdapter™ (PentaBase ApS, Denmark) at 4° C. overnight.

(3) Target Enrichment, Sequencing, and Sequence Data Processing

A RNA bait pool to target about ˜499 kb of the human genome, including exons from 83 cancer-related genes described in Table 1 below, was prepared. Eight purified libraries were pooled and adjusted to a total of 750 ng for each hybrid selection reaction. Target enrichment was performed following the SureSelect bait hybridization protocol with the modification of replacing the blocking oligonucleotide with IDT x Gen blocking oligonucleotide (IDT, Santa Clara, Calif., U.S.A.) for the pre-indexed adapter.

After the target enrichment, the captured DNA fragments were amplified via PCR reactions using P5 and P7 oligonucleotides. The amplified library was purified with AMPure beads and quantified by Picogreen fluorescence assay using a dsDNA HS assay kit and a Qubit 2.0 fluorometer. The fragment size distribution was analyzed using a 2100 Bioanalyzer (Agilent Technologies). Based on DNA concentration and average fragment size, the libraries were normalized to a concentration of 2 nM and pooled by equal volume. After DNA was denatured using 0.2 N NaOH, the denatured libraries were diluted to 20 pM with a hybridization buffer (Illumina, San Diego, Calif., U.S.A.). Cluster amplification of denatured templates was performed according to the manufacturer's protocol (Illumina). Flow cells were sequenced in the 100-bp paired-end mode using the HiSeq 2500 v3 Sequencing-by-Synthesis kits (Illumina) and then analyzed using RTA software (v.1.12.4.2 or later). Using BWA-mem (v0.7.5), all raw data were aligned to the hg19 human reference to create BAM files. SAMTOOLS (v0.1.18), Picard (v.93), and GATK (v3.1.1) were used for sorting SAM/BAM files, followed by local realignments and duplicate markings, respectively. Through the process, duplicates, discordant pairs, and off-target reads were removed.

TABLE 1

ABL1
AKT1
AKT2
AKT3
ALK
APC
ARID1A
ARID1B
ARID2
ATM
ATRX
AURKA
AURKB
BCL2
BRAF
BRCA1
BRCA2
CDH1
CDK4
CDK6
CDKN2A
CSF1R
CTNNB1
DDR2
EGFR
EPHB4
ERBB2
ERBB3
ERBB4
EWSR1
EZH2
FBXW7
FGFR1
FGFR2
FGFR3
FLT3
GNA11
GNAQ
GNAS
HNF1A
HRAS
IDH1
IDH2
IGF1R
ITK
JAK1
JAK2
JAK3
KDR
KIT
KRAS
MDM2
MET
MLH1
MPL
MTOR
NF1
NOTCH1
NPM1
NRAS
NTRK1
PDGFRA
PDGFRB
PIK3CA
PIK3R1
PTCH1
PTCH2
PTEN
PTPN11
RB1
RET
ROS1
SMAD4
SMARCB1
SMO
SRC
STK11
SYK
TERT
TOP1
TP53
TMPRSS2
VHL

The average total reads generated from the plasma and PBL DNA samples were 56.3×10⁶ and 2,000×10⁶ reads, respectively. Further, the read alignment rate was 87.3% for plasma DNA sample and 93.7% for PBL DNA sample. After excluding PCR duplication from sequencing data, the depths for plasma DNA and PBL DNA samples were 1,964×(1,210-3,069×) and 1,717×(1,042-2,361×) on average, respectively.

(4) Identification of Background Allele in Target Region from Sequencing Data

For each paired set of PBL and plasma DNA samples, a base at a position across the entire target regions was determined to be a background allele when the following conditions were met: (1) the base was a non-reference allele; (2) the position displayed sufficient depth of coverage (i.e., >500×) in the paired PBL and plasma DNA samples; and (3) the frequencies of the bases in both PBL and plasma DNA samples did not indicate a germline variant (i.e., <5%). Since samples from cancer patients were used, the candidate alleles for somatic cancer variants were removed. This removal process was achieved by generating sequencing data for matched fine-needle aspiration (FNA) biopsies obtained from patients with cancer at a time close to that of blood collection, prior to therapeutic treatments. Sequencing libraries for the primary tumors were generated using 200 ng of primary tumor input DNA, and analyzed using HiSeq 2500 as described in (3). The depth of FNA DNA sample after removal of duplication in FNA samples was on average 987.15 (790.32-1476.55×). In a paired set of PBL and plasma DNA samples, (1) a position was excluded when the depth at that position was below 250×, and (2) an allele was excluded when it was present at a frequency greater than 2.5% in the sequencing result of the FNA DNA sample.

(5) Analysis of Base Quality Score of Background Allele

After excluding tumor-derived single nucleotide variants (SNVs) and germline single nucleotide polymorphisms (SNPs), background allele errors generated during the sequencing run were analyzed by analyzing the Phred base quality scores of non-reference background alleles.

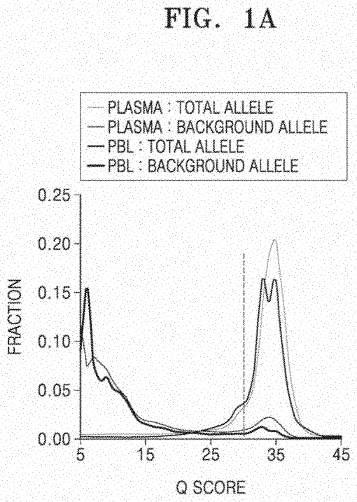

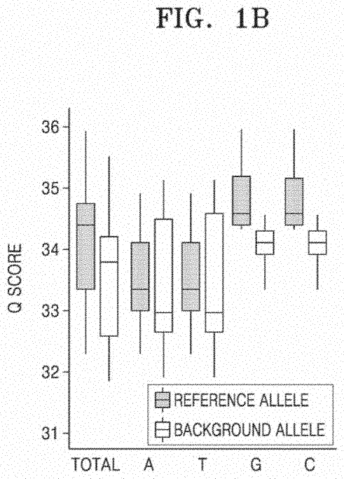

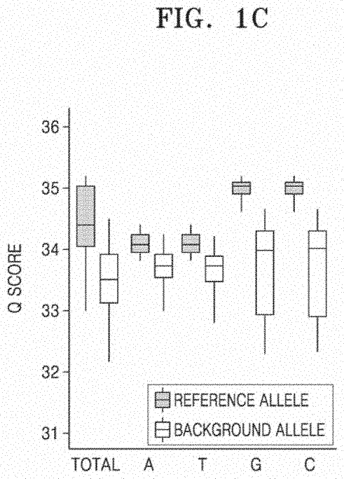

FIG. 1A shows Phred base quality score distribution of background allele bases and total allele bases in PBL DNA samples and plasma DNA samples, respectively. FIG. 1B shows base quality score distribution of each of reference allele bases and background allele bases in PBL DNA samples, after removal of bases with a quality score <30. FIG. 1C shows base quality score distribution of each of reference allele bases and background allele bases in plasma DNA samples, after removal of bases with a quality score <30. As shown in FIGS. 1A to 1C, while most background alleles displayed base quality scores of less than 20, a small fraction of background alleles exhibited a quality score distribution indistinguishable from that of the reference alleles. In the raw sequencing data, the fraction of bases with a quality score ≥30 was 87±3.3% and 87±2.5% for PBL DNA sample and plasma DNA sample, respectively (mean t SD). After the exclusion of bases with a quality score <30, the overall distribution of base quality scores was observed to be not notably different between background and reference alleles. However, the slight differences were observed in the base quality scores for C and G, as a result of A>C and T>G transversions. These results suggest that background allele errors may be generated by other causes than errors incurred during the sequencing run.

(6) Analysis of Background Allele Error Patterns

As described in (5), analysis was performed after excluding errors incurred during the sequencing run by excluding bases with a base quality score <30.

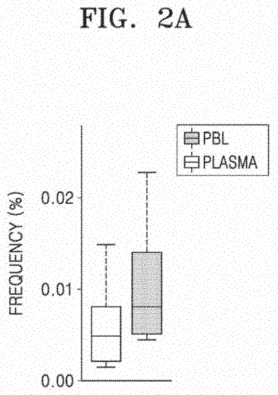

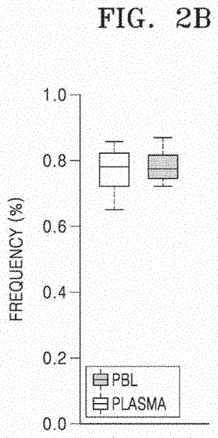

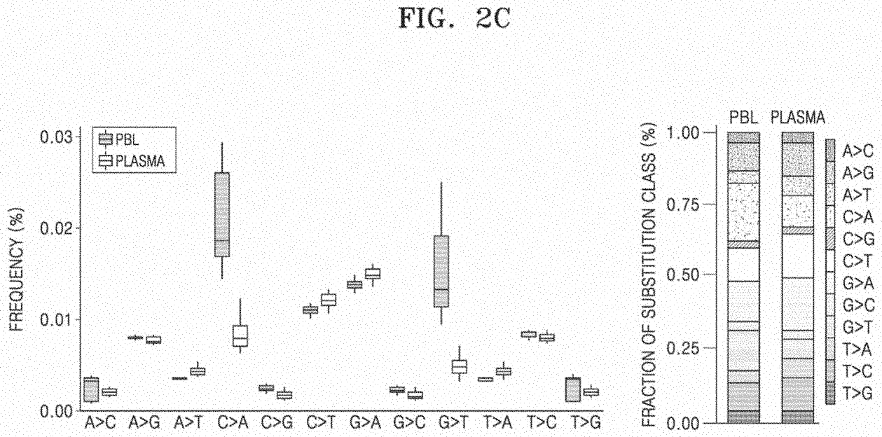

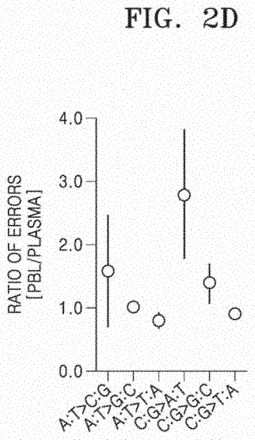

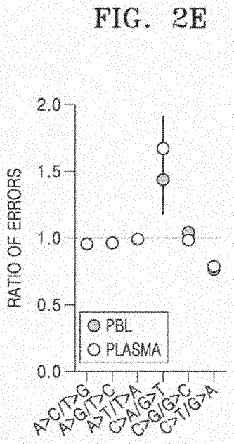

The background allele frequencies were calculated for 19 pairs of plasma DNA samples and PBL DNA samples across the entire target regions. FIG. 2A shows background allele frequencies from 19 plasma DNA samples and 19 PBL DNA samples, i.e., mean error rates of background alleles in each sample. As shown in FIG. 2A, the mean background allele frequency was 0.007 and 0.008% in plasma DNA samples and PBL DNA samples, respectively. FIG. 2B shows frequency of background allele error-free positions in plasma DNA samples and PBL DNA samples. As shown in FIG. 2b, error-free positions were shown to occur at a frequency of 77.2 t 1.4% (mean±SD) for plasma DNA samples and 78.7 t 1.0% for PBL DNA samples across the entire target regions. FIG. 2C shows a distribution of background allele frequencies across 12 base substitution classes in plasma DNA samples and PBL DNA samples. The y-axis represents the background allele frequency of each substitution class in the pre-treatment PBL DNA samples and plasma DNA samples. FIGS. 2D and 2E show background allele error rates for 12 base substitution classes in plasma DNA samples and PBL DNA samples. As shown in FIGS. 2C to 2E, C:G>A:T nucleotide transversion showed a significant difference between plasma DNA samples and PBL DNA samples. In particular, among stall the nucleotide substitutions, C:GA:T and C:G>G:C transversion errors were significantly increased in PBL DNA samples, as compared to plasma DNA samples.

2. Change of Fragmentation Conditions of Nucleic Acid Isolated from Cells and Influence of Nucleic Acid Fragmentation Conditions on Background Allele Error Rates

The background allele error rates were analyzed in the same manner as in 1, except that energy intensity and/or duration of DNA fragmentation were/was varied to test whether DNA fragmentation influenced the background allele error rate.

Detailed fragmentation conditions are the same as in the following Table.

TABLE 2

| Conditions | A | B | C | D |
|---|---|---|---|---|
| Duty factor | 10% | 10% | 5% | 5% |
| Peak incident power (W) | 175 | 140 | 105 | 105 |
| (peak incident power) | | | | |
| Cycles per burst | 200 | 200 | 200 | 200 |
| (Cycles per burst) | | | | |
| Time (sec) | 350 | 80 | 80 | 50 |
| Volume (μl) | 50 | 50 | 50 | 50 |
| Temperature (° C.) | 4-7 | 4-7 | 4-7 | 4-7 |
| Water volume (μl) | 12 | 12 | 12 | 12 |
| Median fragment size | 170 | 320 | 425 | 490 |
| (nt) | | | | |
| (Median fragment size) | | | | |

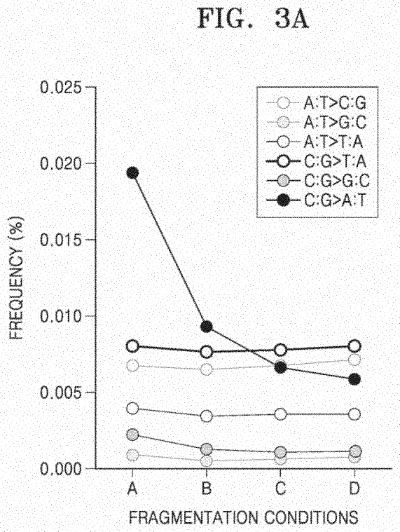

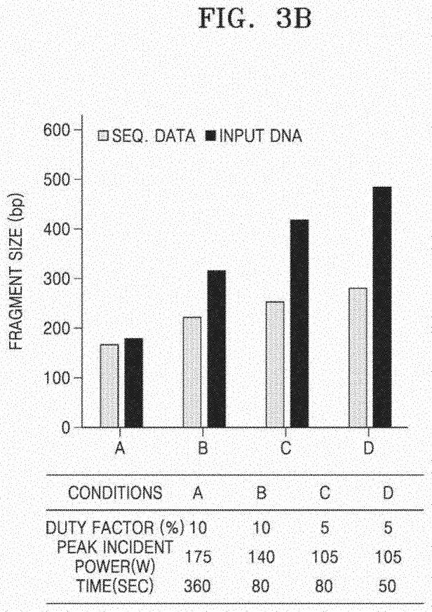

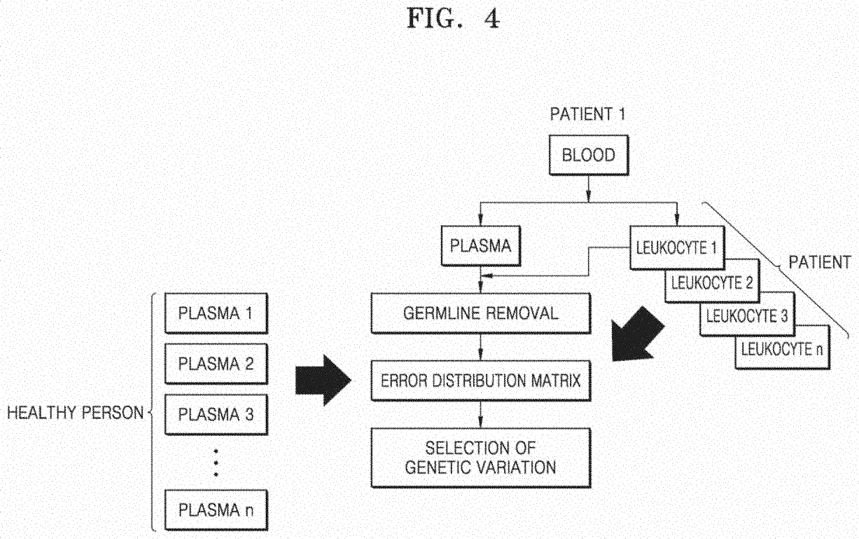

FIG. 3A shows background allele error rats from sequencing data generated using genomic DNA fragments as input DNA, wherein the genomic DNA fragments were obtained by fragmentation under various fragmentation conditions. FIG. 3B shows detailed conditions of the fragmentation conditions used in FIG. 3A and the sizes of the resulting fragments. As shown in FIG. 3A, when relatively low energy was applied during fragmentation, the rates of C:G>A:T and C:G>G:C transversions in PBL DNA samples were decreased to match the rates of C:G>A:T and C:G>G:C transversions in plasma DNA samples. As shown in FIG. 3B, when relatively low energy was applied during fragmentation, the size of input DNA was increased. However, the increase in the sizes of DNA inserts for sequencing was small, compared to the increase in the size of the input DNA.

In other words, since the DNA fragmentation step causes damage to induce C:G>A:T and C:G>G:C transversions, background allele error rates may be reduced by lowering energy consumed for fragmentation of nucleic acids isolated from cells, and thus the distribution of background allele frequency of nucleic acids isolated from cells was similar to that of cell-free nucleic acids. Accordingly, it is possible to accurately detect rare variations without using sequencing data obtained from nucleic acids of a normal person.

Hereinabove, the present disclosure has been described with reference to exemplary embodiments thereof. Therefore, it will be understood by those skilled in the art to which the present disclosure pertains that the present disclosure may be implemented in modified forms without departing from the spirit and scope of the present disclosure. Therefore, exemplary embodiments disclosed herein should be considered in an illustrative aspect rather than a restrictive aspect. The scope of the present disclosure should be defined by the claims rather than the above-mentioned description, and it shall be interpreted that all differences within the equivalent scope are included in the present disclosure.

1. A method of generating a distribution of background allele frequency in sequencing data obtained from a cell-free nucleic acid, comprising:
obtaining first sequencing data of one or more positions on a chromosome from the cell-free nucleic acid;
obtaining second sequencing data of one or more positions on the chromosome from a nucleic acid isolated from a cell;
generating a distribution of background allele frequency at one or more positions on the chromosome, based on the second sequencing data; and
estimating the distribution of background allele frequency in the first sequencing data using the distribution of background allele frequency.

2. The method of claim 1, comprising performing fragmentation of the nucleic acid isolated from the cell, prior to obtaining the second sequencing data.

3. The method of claim 2, wherein the fragmentation is physical, chemical, thermal, optical, ultrasonic, or enzymatic cleavage of the nucleic acid isolated from the cell.

4. The method of claim 3, wherein the ultrasonic cleavage is applying ultrasonic waves at 50 W to 160 W for 10 seconds to 300 seconds.

5. The method of claim 2, wherein the sizes of the fragmented nucleic acids are 200 bp or more.

6. The method of claim 1, wherein the nucleic acid isolated from the cell and the cell-free nucleic acid are derived from the same subject or different subjects.

7. The method of claim 1, wherein the nucleic acid isolated from the cell is isolated from a blood cell, an oral epithelial cell, a hair follicle cell, a skin fibroblast, or a combination thereof.

8. The method of claim 1, wherein the cell-free nucleic acid is present in blood, plasma, serum, urine, saliva, mucous secretions, sputum, feces, tears, or a combination thereof.

9. The method of claim 1, wherein the cell-free nucleic acid is a circulating tumor nucleic acid.

10. A frequency distribution matrix of background alleles in sequencing data obtained from a cell-free nucleic acid, generated by:
obtaining first sequencing data of one or more positions on a chromosome from the cell-free nucleic acid;
obtaining second sequencing data of one or more positions on the chromosome from a nucleic acid isolated from a cell;
generating a distribution of background allele frequency at one or more positions on the chromosome, based on the second sequencing data; and
estimating the distribution of background allele frequency in the first sequencing data using the distribution of background allele frequency.

11. A method of detecting a variation in a cell-free nucleic acid, comprising:
obtaining first sequencing data of one or more positions on a chromosome from the cell-free nucleic acid;
obtaining second sequencing data of one or more positions on the chromosome from a nucleic acid isolated from a cell;
generating a distribution of background allele frequency at one or more positions on the chromosome, based on the second sequencing data; and
detecting variations by comparing any allele frequency at one or more positions on the chromosome in the first sequencing data with the distribution of background allele frequency at positions corresponding thereto.

12. The method of claim 11, comprising:
determining that the allele is a significant variant when any allele frequency at one or more positions on the chromosome in the first sequencing data is larger than the distribution of background allele frequency at the positions corresponding thereto; and
determining that the allele is not a significant variant when any allele frequency at one or more positions on the chromosome in the first sequencing data is smaller than or equal to the distribution of background allele frequency at the positions corresponding thereto.
